(19) United States
(12) Patent Application Publication
(10) Pub. No.: US 2017/0112740 A1
(43) Pub. Date: Apr. 27, 2017

(54) DECOLORIZATION OF DYED KERATIN FIBERS

(71) Applicant: Henkel AG & Co. KGaA, Duesseldorf (DE)

(72) Inventors: Juergen Schoepgens, Schwalmtal (DE); Burkhard Mueller, Duesseldorf (DE)

(73) Assignee: Henkel AG & Co. KGaA, Duesseldorf (DE)

(21) Appl. No.: 15/399,683

(22) Filed: Jan. 5, 2017

Related U.S. Application Data
(63) Continuation of application No. PCT/EP2015/063168, filed on Jun. 12, 2015.

(30) Foreign Application Priority Data
Jul. 9, 2014 (DE) 10 2014 213 318.1

(51) Int. Cl. A61K 8/46; A61K 8/02; A61Q 5/08; A61K 8/23

(52) U.S. Cl. CPC A61K 8/466; A61K 8/23; A61K 8/0216; A61Q 5/08; A61K 2800/262; A61K 2800/88; A61K 2800/882

(57) ABSTRACT

The invention relates to agents for the reductive decolorization of dyed keratin fibers, in particular human hair, containing in an aqueous cosmetic carrier (a) one or more reduction agents from the group consisting of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, sulfanylacetic acid (thioglycolic acid), and/or ascorbic acid, and (b) one or more zwitterionic surfactants, each of which has at least one quaternary ammonium group and a grouping of —SO₃— as structural units.

### FIELD OF THE INVENTION

The present invention generally relates to compositions for the reductive removal of color from dyed keratinic fibers, in particular human hair, containing in a cosmetic carrier at least one reduction agent and at least one zwitterionic surfactant, each of which has a quaternary ammonium group and a grouping of —SO₃—. A multi-component packaging unit (kit-of-parts) containing the previously described compositions and a method for decolorizing keratin fibers are also subjects of the invention. The present invention also comprises the use of zwitterionic surfactants, each of which has a quaternary ammonium group and a grouping of —SO₃— for improving the decolorization effect of reduction agents with the reductive removal of color from keratinic fibers.

### BACKGROUND OF THE INVENTION

Preparations for tinting and dyeing hair are an important type of cosmetic agent. They can be used to lightly or heavily nuance the natural hair color in accordance with the wishes of the person in question, to attain a completely different hair color or to cover undesirable shades of color, for example grey shades. Conventional hair colorants, depending on the desired color or permanence of the coloration, are formulated either on the basis of oxidation dyes or on the basis of substantive dyes. Combinations of oxidation dyes and substantive dyes are also often used to attain specific nuances.

Colorants based on oxidation dyes lead to bright and permanent color shades. However, they require the use of strong oxidizing agents, such as hydrogen peroxide solutions. Such colorants include oxidation dye precursors, or what are known as developer components and coupler components. The developer components form the actual dyes under the influence of oxidizing agents or atmospheric oxygen, either among themselves or with coupling to one or more coupler components.

Colorants based on substantive dyes are often used for temporary coloring. Substantive dyes are dye molecules which are drawn directly onto the hair and do not require any oxidative process to form the color. Important representatives of this class of dyes include, for example, triphenylmethane dyes, azo dyes, anthracine dyes, or nitrobenzene dyes, which can each carry cationic or anionic groups.

In all of these dyeing processes, it may be that the coloring must be reversed again wholly or partially for various reasons. A partial removal of the coloring is suggested for example when the color result on the fibers is darker than desired.

On the other hand, a complete removal of the coloring may also be desired in some cases. By way of example, it is thus conceivable for the hair to be dyed or tinted in a specific nuance for a certain reason and then for the original color to be restored again after a few days.

Agents and methods for dye removal are already known in the literature. A method that has long been known from the prior art for reversing coloring is the oxidative decolorization of the dyed hair, for example with the aid of a conventional bleaching agent. In this process, however, the fibers can be damaged by the use of strong oxidizing agents.

Furthermore, reductive processes for dye removal have also already been described. By way of example, European patent application EP 1300136 A2 discloses methods for hair treatment in which the hair is dyed in a first step and is reductively decolorized again in a second step. Here, the reductive decolorization occurs as a result of the application of a formulation containing a dithionite salt and a surfactant. In WO 2008/055756 A2 the reductive decolorization of keratin fibers is performed by means of a mixture of a reduction agent and an absorption agent.

With use of reductive decolorizing agents, the decolorization takes place by reduction of the dyes located on the keratin fibers or hairs. As a result of the reduction, the dyes are generally converted into their reduced leukoforms. During this process, the double bonds present in the dyes are reduced, the chromophoric system of the dyes is thus interrupted, and the dye is converted into a colorless form.

A general problem with the reductive decolorizing agents known from the prior art lies in the fact that the dyed keratin fibers can indeed be decolorized initially by use of the reduction agent, however the color removal is not permanent. In particular in the case of oxidatively dyed hair, in which the coloring is produced by oxidation dye precursors of the developer and of the coupler type, colorings are obtained that have very good fastness properties in part. With use of the reductive decolorizing agent, these dyes are now reductively converted into uncolored compounds—these still remain on the hair, however, on account of similarly good fastness properties.

Once the reduction agent has been rinsed off and under the influence of atmospheric oxygen, these reduced forms can now be re-oxidized again little by little. A lesser or greater re-coloration takes place on account of this re-oxidation. This re-coloration generally does not correspond to the shade to which the keratin fibers were dyed previously, and instead may be unattractive at random points and is therefore even less desired by the user of the decolorizing agent.

The object of the present invention was therefore to provide a decolorizing agent for the decolorization of dyed keratinic fibers which decolorizes dyed keratin fibers as completely as possible. The decolorization should be long lasting, and the decolorized keratin fibers should not suffer from any re-coloration, nuance shift, or darkening under the influence of atmospheric oxygen. The decolorizing agent should, in particular, demonstrate a good decolorization effect on the keratin fibers dyed previously with oxidative colorants on the basis of oxidation dye precursors of the developer and of the coupler type.

It has surprisingly now been found that this object can be achieved by the use of decolorizing agents which include one or more inorganic reduction agents and at least one specific zwitterionic surfactant in an aqueous cosmetic carrier. The specific zwitterionic surfactants are characterized in that they each have at least one quaternary ammonium group and a grouping of —SO₃— as structural units.

Furthermore, other desirable features and characteristics of the present invention will become apparent from the subsequent detailed description of the invention and the appended claims, taken in conjunction with this background of the invention.

### BRIEF SUMMARY OF THE INVENTION

An agent for the reductive decolorization of dyed keratinic fibers, in particular human hair, containing in an aqueous cosmetic carrier one or more reduction agents from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, thioglycolic acid and/or ascorbic acid; and one or more zwitterionic surfactants, each of which has at least one quaternary ammonium group and at least one grouping of —SO₃— as structural units.

Use of a zwitterionic surfactant which has at least one quaternary ammonium group and one grouping of —SO₃— as structural units to improve the decolorizing effect of one or more reduction agents from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, sodium disulfite, potassium disulfate, ammonium disulfite, hydroxymethanesulfinic acid, aminomethanesulfinic acid, cysteine, thiolactic acid, thioglycolic acid and/or ascorbic acid in the reductive decolorization of dyed keratinic fibers.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject matter of the present invention is an agent for the reductive decolorization of dyed keratinic fibers, in particular human hair, containing in an aqueous cosmetic carrier

(a) one or more reduction agents from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, thioglycolic acid and/or ascorbic acid, and
(b) one or more zwitterionic surfactants, each of which has at least one quaternary ammonium group and at least one grouping of —SO₃— as structural units.

The use of the decolorizing agents according to the invention causes—depending on the choice of dyes previously used for coloring—a nearly complete or even complete decolorization of previously dyed keratin fibers. The decolorizing agent according to the invention shows an excellent effect especially on keratin fibers previously dyed with oxidation dyes. It has been found particularly surprisingly that this decolorization effect lasts even once the reduction agent has been rinsed out, and that the decolorized keratin fibers, which are exposed to the action of atmospheric oxygen for hours or days, do not suffer any re-oxidation or darkening.

Keratinic fibers, keratin-containing fibers or keratin fibers are to be understood to mean furs, wool, feathers and in particular human hair. Although the agents according to the invention are primarily suitable for decolorization of keratin fibers or human hair, there is nothing in principle standing in the way of a use in other fields as well.

The term “dyed keratinic fibers” is understood to mean keratin fibers that have been dyed using conventional cosmetic colorants known to a person skilled in the art. In particular, “dyed keratinic fibers” are understood to mean fibers which have been dyed using the oxidative colorants known from the prior art and/or using substantive dyes. Express reference is made in this regard to the known monographs, for example Kh. Schrader, Grundlagen and Rezepturen der Kosmetika (Basic Principles and Formulations of Cosmetics), 2^{nd} edition, Hüthig Buch publishers, Heidelberg, 1989, which reflect the corresponding knowledge of a person skilled in the art.

The agents include each of the ingredients essential to the invention in an aqueous cosmetic carrier, for example in a suitable aqueous or aqueous-alcoholic carrier. For the purposes of the reductive decolorization, such carriers can be, for example, creams, emulsions, gels or also surfactant-containing foaming solutions, such as shampoos, foam aerosols, foam formulations or other preparations suitable for use on hair. Agents for reductive color removal from keratinic fibers are particularly preferably creams, emulsions or flowable gels.

Reduction Agent (a)

As first ingredient (a) essential to the invention, the decolorizing agents include one or more reduction agents from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, sodium disulfite, potassium dislufite, ammonium disulfite, hydroxymethanesulfinic acid, aminomethanesulfinic acid, cysteine, thiolactic acid, thioglycolic acid (alternative name: sulfanylacetic acid) and/or ascorbic acid.

Sodium dithionite is an inorganic reduction agent having the empirical formula Na₂S₂O₄ and the CAS number 7775-14-6.

Zinc dithionite is an inorganic reduction agent having the empirical formula Zn₂S₂O₄ and the CAS number 7779-86-4.

Potassium dithionite is an inorganic reduction agent having the empirical formula K₂S₂O₄ and the CAS number 14293-73-3.

Sodium sulfite is an inorganic reduction agent having the empirical formula Na₂SO₃ and the CAS number 7757-83-7.

Sodium hydrogen sulfite is an inorganic reduction agent having the empirical formula NaHSO₃ and the CAS number 7631-90-5. Sodium hydrogen sulfite is preferably used in the form of an aqueous solution.

Potassium sulfite is an inorganic reduction agent having the empirical formula K₂SO₃ and the CAS number 10117-38-1.

Potassium hydrogen sulfite is an inorganic reduction agent having the empirical formula KHSO₃ and the CAS number 7773-03-7.

Ammonium sulfite is an inorganic reduction agent having the empirical formula (NH₄)₂SO₃ and the CAS number 10196-04-0.

Sodium thiosulfate is an inorganic reduction agent having the empirical formula Na₂S₂O₃ and the CAS number 7772-98-7.

Potassium thiosulfate is an inorganic reduction agent having the empirical formula K₂S₂O₃ and the CAS number 10294-66-3.

Ammonium thiosulfate is an inorganic reduction agent having the empirical formula (NH₄)₂S₂O₃ and the CAS number 7783-18-8.

Hydroxymethanesulfinic acid is an organic reduction agent having the empirical formula HO—CH₂—S(O)OH and the CAS number 79-25-4. Alternatively, hydroxymethanesulfinic acid is also referred to as formaldehyde sulfoxyl acid. Both hydroxymethanesulfinic acid itself and the physiologically acceptable salts of hydroxymethanesulfinic acid, for example the sodium salt and/or the zinc salt, can be used in accordance with the invention. The use of sodium formaldehyde sulfoxylate (sodium hydroxymethanesulfinate, the sodium salt of hydroxymethanesulfinic acid) and/or zinc formaldehyde sulfoxylate (zinc hydroxymethanesulfinate, the zinc salt of sodium hydroxymethanesulfinate) is therefore also possible in accordance with the invention.

Aminomethanesulfinic acid is an organic reduction agent having the formula H₂N—CH₂—S(O)OH and the CAS number 118201-33-5. Both aminomethanesulfinic acid itself and the physiologically acceptable salts of aminomethanesulfinic acid, for example the sodium salt and/or the zinc salt, can be used in accordance with the invention. The use of sodium aminomethanesulfinate (the sodium salt of aminomethanesulfinic acid) and/or zinc aminomethanesulfinate (the zinc salt of sodium aminomethanesulfinate) is therefore also possible in accordance with the invention.

Cysteine (2-amino-3-suflanylpropionic acid) is understood in accordance with the invention to mean D-cysteine, L-cysteine and/or a mixture of D- and L-cysteine.

Thiolactic acid (2-sulfanylpropionic acid) is understood to mean D-thiolactic acid, L-thiolactic acid and/or a mixture of D- and L-thiolactic acid. Both thiolactic acid itself and thiolactic acid in the form of a physiologically acceptable salt thereof can be used in accordance with the invention. A preferred salt of thiolactic acid is ammonium thiolactate.

Ammonium thiolactate is the ammonium salt of thiolactic acid (i.e. the ammonium salt of 2-sulfanylpropionic acid) (formula XX).

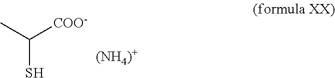

The definition of ammonium thiolactate includes both the ammonium salts of D-thiolactic acid and the ammonium salts of L-thiolactic acid, and mixtures thereof

Thioglycolic acid (sulfanylacetic acid, 2-mercaptoacetic acid) is understood to be an organic reduction agent of the formula HS—CH₂—COOH, and the compound has the CAS number 68-11-1. In the case of thioglycolic acid as well, thioglycolic acid itself and a physiologically acceptable salt of thioglycolic acid can be used in accordance with the invention. By way of example, sodium thioglycolate, potassium thioglycolate and/or ammonium thioglycolate can be used as physiologically acceptable salts of thioglycolic acid. Ammonium thioglycolate is a preferred physiologically acceptable salt of thioglycolic acid.

Ammonium thioglycolate is the ammonium salt of thioglycolic acid (i.e. the ammonium salt of sulfanylacetic acid) formula (XXX).

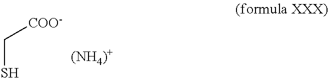

Ascorbic acid is understood in accordance with the invention to mean, in particular, (R)-5-[(S)-1,2-dihydroxyethyl]-3,4-dihydroxy-5H-furan-2-one (further alternative names: vitamin C, L-ascorbic acid) having the CAS number 50-81-7.

The reduction agents sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate and/or ammonium thiosulfate have proven to be particularly well suited for reductive decolorization. If these aforementioned preferred reduction agents (a) are used in combination with the zwitterionic surfactants (b) according to the invention, a particularly effective decolorization and a particularly effective prevention of re-oxidation in the decolorized strands of hair can be observed. In this way, a darkening of the decolorized keratin fibers can be prevented over a particularly long period of time.

Furthermore, the reduction agent(s) from group (a) is/are preferably used in specific amount ranges. A decolorizing effect is observed with just small amounts. In order to obtain a sufficient and strong decolorization effect, however, it is preferred when the decolorizing agent includes the reduction agent(s) (a) in a total amount of from 0.5 to 20.5% by weight, preferably from 3.5 to 15.5% by weight, more preferably from 6.0 to 13.5% by weight, and particularly preferably from 7.5 to 11.5% by weight, in relation to the total weight of the agent.

A preferred agent for the reductive decolorization of dyed keratinic fibers, in particular human hair, is therefore one containing in an aqueous cosmetic carrier

(a) one or more reduction agents from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, thioglycolic acid and/or ascorbic acid in a total amount of from 0.5 to 20.5% by weight, preferably from 3.5 to 15.5% by weight, more preferably from 6.0 to 13.5% by weight, and particularly preferably from 7.5 to 11.5% by weight, in relation to the total weight of the agent, and
(b) one or more zwitterionic surfactants, each of which has at least one quaternary ammonium group and a grouping of —SO₃— as structural units.

A very particularly preferred agent for the reductive decolorization of dyed keratinic fibers is also characterized in that it includes

(a) one or more reduction agents from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate and/or ammonium thiosulfate in a total amount of from 0.5 to 20.5% by weight, preferably from 3.5 to 15.5% by weight, more preferably from 6.0 to 13.5% by weight, and particularly preferably from 7.5 to 11.5% by weight, in relation to the total weight of the agent.

It has also proven to be particularly advantageous when the agents according to the invention include combinations of reduction agents from the group (a), since specific combinations result in a very particularly strong decolorization effect. In this regard, the use of two different reduction agents from group (a) is particularly advantageous, wherein the decolorizing agent includes

(a1) a first reduction agent selected from the group of sodium dithionite, zinc dithionite, potassium dithionite, sodium thiosulfate, potassium thiosulfate and/or ammonium thiosulfate, and additionally
(a2) a second reduction agent selected from the group of sodium sulfite, sodium hydrogen sulfite, potassium sulfite, potassium hydrogen sulfite and/or ammonium sulfite.

In other words, particularly preferred agents for the reductive decolorization of dyed keratinic fibers, in particular human hair, are those that include in an aqueous cosmetic carrier

(a1) a first reduction agent selected from the group of sodium dithionite, zinc dithionite, potassium dithionite, sodium thiosulfate, potassium thiosulfate and/or ammonium thiosulfate, and additionally
(a2) a second reduction agent selected from the group of sodium sulfite, sodium hydrogen sulfite, potassium sulfite, potassium hydrogen sulfite and/or ammonium sulfite, and
(b) one or more zwitterionic surfactants, each of which has a quaternary ammonium group and a grouping of —SO₃— as structural units.

Zwitterionic Surfactants (b)

As second constituent (b) essential to the invention, the decolorizing agents include one or more zwitterionic surfactants, each of which has a quaternary ammonium group and a grouping of —SO₃— as structural units.

Surfactants are understood to mean amphiphilic (bifunctional) compounds comprising at least one hydrophobic group and at least one hydrophilic molecule part. The hydrophobic molecule part is usually a hydrocarbon chain having 10 to 30 carbon atoms.

In the case of the zwitterionic surfactants, the hydrophilic molecule part comprises a zwitterionic structural unit, i.e. a structural unit which comprises both a cationically charged and an anionically charged molecule part. Zwitterionic surfactants (b) according to the invention are characterized in that they have a cationically charged molecule part in the form of a quaternary ammonium group and their anionic molecule part is present in the form of a grouping of —SO₃—.

An ammonium group is quaternary when a grouping of the (R₁R₂R₃R₄N)⁺ type is present, i.e. when all four H atoms of the NH₄ ion from which the quaternary ammonium group is derived, are replaced by organic groups R (or R1 to R4).

The grouping of —SO₃— of the zwitterionic surfactant can be present bonded directly to a carbon atom. In this case the anionic part of the zwitterionic compound is a deprotonated sulfonic acid grouping of the formula R₅R₆R₇C—SO₃—; here, the groups R5, R6, R7 constitute the remaining part of the zwitterionic surfactant according to the invention.

However, the invention also includes the case in which the grouping of —SO₃— is bonded to a carbon atom via an oxygen atom. In this case the anionic part of the zwitterionic surfactant constitutes a deprotonated sulfuric acid ester of formula R₅R₆R₇C—SO₃—, wherein the groups R5, R6, R7 again constitute the remaining part of the zwitterionic surfactant according to the invention.

It is preferred in accordance with the invention when the grouping of —SO₃— is directly bonded to a carbon atom, i.e. is present in the form of a deprotonated sulfonic acid grouping.

A darkening of the decolorized keratin fibers can be prevented over a particularly long period of time when at least one compound of formula (I) is used as zwitterionic surfactant (b) according to the invention,

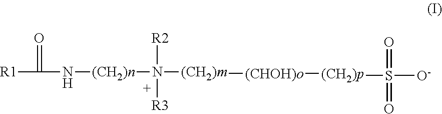

in which
R1 stands for a linear or branched C₉-C₂₉ alkyl group, a linear or branched C₉-C₂₉ alkenyl group, or a linear or branched hydroxy C₉-C₂₉ alkyl group,
R2 and R2 independently of one another, stand for a C₁-C₆ alkyl group, a C₂-C₆ alkenyl group, or a hydroxy C₂-C₆ alkyl group,
n stands for an integer from 1 to 6,
m stands for an integer from 0 to 6,
o stands for an integer from 0 to 6,
p stands for an integer from 0 to 6,
with the provision that the sum of m, o and p is at least 1.

In a further embodiment a very particularly preferred agent for the reductive decolorization of dyed keratinic fibers is therefore one containing

(a) one or more reduction agents from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate and/or ammonium thiosulfate, and
(b) one or more zwitterionic surfactants f formula (I),

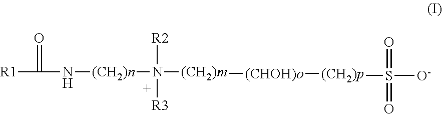

in which
R1 stands for a linear or branched C₉-C₂₉ alkyl group, a linear or branched C₉-C₂₉ alkenyl group, or a linear or branched hydroxy C₉-C₂₉ alkyl group,
R2 and R3 independently of one another, stand for a C₁-C₆ alkyl group, a C₂-C₆ alkenyl group, or a hydroxy C₂-C₆ alkyl group,
n stands for an integer from 1 to 6,
m stands for an integer from 0 to 6,
o stands for an integer from 0 to 6,
p stands for an integer from 0 to 6,
with the provision that the sum of m, o and p is at least 1.

The use of the zwitterionic surfactants (b) according to the invention prevents the darkening of the decolorized strands and thus ensures a long-lasting decolorization effect. Here, the use of the surfactants in specific amount ranges is of particular advantage. It is particularly preferred when the zwitterionic surfactant(s) (b) is/are used in the decolorizing agent according to the invention in a total amount of from 0.1 to 15.0% by weight, preferably from 0.5 to 12% by weight, more preferably from 1.0 to 9.0% by weight, and particularly preferably from 2.0 to 6.0% by weight, in relation to the total weight of the agent.

In a further very particularly preferred embodiment, a decolorizing agent according to the invention is therefore characterized in that it includes, as zwitterionic surfactant (b), one or more surfactants of formula (I) in a total amount of from 0.1 to 15.0% by weight, preferably from 0.5 to 12% by weight, more preferably from 1.0 to 9.0% by weight, and particularly preferably from 2.0 to 6.0% by weight, in relation to the total weight of the agent,

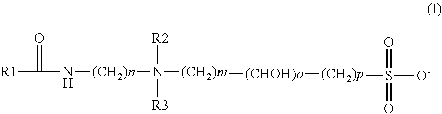

in which
R1 stands for a linear or branched C₉-C₂₉ alkyl group, a linear or branched C₉-C₂₉ alkenyl group, or a linear or branched hydroxy C₉-C₂₉ alkyl group,
R2 and R3 independently of one another, stand for a C₁-C₆ alkyl group, a C₂-C₆ alkenyl group, or a hydroxy C₂-C₆ alkyl group,
n stands for an integer from 1 to 6,
m stands for an integer from 0 to 6,
o stands for an integer from 0 to 6,
p stands for an integer from 0 to 6,
with the provision that the sum of m, o and p is at least 1.

The use of zwitterionic surfactants of formula (I) in which the group R1 stands for a linear C₁₁ alkyl group, a linear C₁₃ alkyl group, a linear C₁₅ alkyl group, a linear C₁₇ alkyl group, or a linear C₁₉ alkyl group, a linear monounsaturated C₁₁ alkenyl group, a linear monounsaturated C₁₃ alkenyl group, a linear monounsaturated C₁₇alkenyl group, or a linear monounsaturated C₁₉ alkenyl group is particularly preferred.

It is also very particularly preferred when n stands for the number 3.

The groups R2 and R3 preferably stand, independently of one another, for a C₁-C₆ alkyl group, and R2 and R3 particularly preferably stand for a methyl group.

m stands very particularly preferably for the numbers 1 or 2.

o stands very particularly preferably for the number 1.

p stands very particularly preferably for the numbers 1 or 2.

In a further very particularly preferred embodiment, a decolorizing agent according to the invention is characterized in that it includes, as zwitterionic surfactant (b), one or more surfactants of formula (I) in a total amount of from 0.1 to 15.0% by weight, preferably from 0.5 to 12% by weight, more preferably from 1.0 to 9.0% by weight, and particularly preferably from 2.0 to 6.0% by weight, in relation to the total weight of the agent,

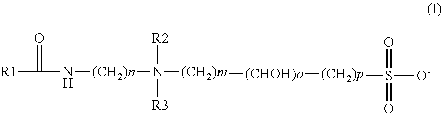

in which
R1 stands for a linear C₁₁ alkyl group, a linear C₁₃ alkyl group, or a linear C₁₅ alkyl group, a linear C₁₇ alkyl group, a linear C₁₉ alkyl group, a linear monounsaturated C₁₁ alkenyl group, a linear monounsaturated C₁₃ alkenyl group, a linear monounsaturated C₁₇-alkenyl group, or a linear monounsaturated C₁₉ alkenyl group,
R2 and R3 both stand for a methyl group,
n stands for an integer from 1 to 3,
m stands for the number 1 or 2,
o stands for the number 1,
p stands for the number 1 or 2.

The zwitterionic surfactants of this very particularly preferred type are also known under the name “amidopropyl hydroxysultaine”:

Laurylamidopropyl hydroxysultaine is a zwitterionic surfactant of formula (I), in which R1 stands for C₁₁ alkyl, R2 stands for methyl, R3 stands for methyl, n stands for 3, m stands for 1, o stands for 1, and p stands for 1.

Cocoamidopropyl hydroxysultaine is a mixture of compounds of formula (I) in which R1 stands for C₁₁ alkyl to C₁₇ alkyl, R2 stands for methyl, R3 stands for methyl, n stands for 3, m stands for 1, o stands for 1, and p stands for 1.

Oleamidopropyl hydroxysultaine is a zwitterionic surfactant of formula (I), in which R1 stands for a monounsaturated C₁₇ alkenyl group (the double bond lies between atoms 8 and 9 of the R1 group), R2 stands for methyl, R3 stands for methyl, n stands for 3, m stands for 1, o stands for 1, and p stands for 1.

Cocoamidopropyl hydroxysultaine is explicitly very particularly preferably used, as can be purchased for example under the trade names Mirataine CBS from the company Rhodia, A mixture of zwitterionic surfactants (b) of formula (I) is therefore very particularly preferably used in accordance with the invention, in which mixture R1 stands for CH alkyl to C₁₇ alkyl, R2 stands for methyl, R3 stands for methyl, n stands for 3, m stands for 1, o stands for 1, and p stands for 1.

The zwitterionic surfactants (b) are used together with the reduction agent (a) in an aqueous cosmetic carrier and thus form the decolorizing agent according to the invention. This decolorizing agent can be set to different pH values by the addition of acids and bases. The grouping of —SO₃— of the surfactants (b) can also be partially protonated in the aqueous cosmetic carrier in the event of a change to the pH value in an equilibrium reaction. These forms of the zwitterionic surfactants protonated by the equilibrium reaction are also included by this invention. All specified amount values relate to the total amount of the zwitterionic surfactants added in their zwitterionic form to the aqueous carrier.

Proportion (a)/(b)

The reduction agent(s) (a) and the zwitterionic surfactant(s) (b) together cause an effective, long-lasting decolorization of dyed keratin fibers. Here, the addition of the specific zwitterionic surfactants with grouping of —SO₃— means that, once the decolorization process is complete, no re-oxidation takes place under the influence of atmospheric oxygen. In this way, a darkening and therefore a subsequent weakening of the decolorization result can be prevented. Since the entire decolorization process thus requires an interaction between the reduction agents (a) and the zwitterionic surfactants (b), the active substances of both categories (a) and (b) are preferably used in specific ratios to one another.

It has been found that the darkening can be particularly effectively prevented when the reduction agents (a) are used at least in the same total amount in comparison to the zwitterionic surfactants (b), but preferably in a surplus. The total amount of the reduction agents (a) is preferably selected to be higher than the total amount of the zwitterionic surfactants (b) by a factor of 1.8, more preferably by a factor of 2.6, and particularly preferably by a factor of 3.2.

Very particularly preferred decolorizing agents are therefore characterized in that the total ratio of the total amount of all reduction agents (a) to the total amount of all zwitterionic surfactants from group (b) included in the agent, i.e. the ratio by weight of (a)/(b), lies at a value of at least 1.0, preferably of at least 1.8, more preferably of at least 2.6, and particularly preferably of at least 3.2.

### Example

100 g of decolorizing agent include 8.0 g of sodium dithionite (total amount of all reduction agents (a)).

With use of 8.0 g of cocamidopropyl hydroxysultaine (active substance), the ratio by weight of (a)/(b) lies at a value of 1.0. With use of 4.4 g of cocamidopropyl hydroxysultaine, the ratio by weight of (a)/(b) lies at a value of approximately 1.8. With use of 3.1 g of cocamidopropyl hydroxysultaine, the ratio by weight of (a)/(b) lies at a value of approximately 2.6. With use of 2.5 g of cocamidopropyl hydroxysultaine, the ratio by weight of (a)/(b) lies at a value of approximately 3.2.

Although the use of a weight surplus of reduction agent (a) has proven to be advantageous, the amount of zwitterionic surfactants (b) used should also not be selected to be too small, so that the darkening of the decolorized strands can be inhibited to a sufficient extent. It is furthermore particularly advantageous when the total amount of reduction agents (a) included in the agent exceeds the total amount of the zwitterionic surfactants (b) at most by a factor of 7.0, preferably at most by a factor of 6.2, more preferably at most by a factor of 5.6, and particularly preferably at most by a factor of 4.8.

Very particularly preferred decolorizing agents are therefore characterized in that the ratio by weight of the total amount of all reduction agents from the group (a) included in the agent to the total amount of all zwitterionic surfactants from the group (b) included in the agent, i.e. the ratio by weight of (a)/(b), lies at a value of at most 7.0, preferably of at most 6.2, more preferably of at most 5.6, and particularly preferably of at most 4.8.

### Example

100 g of decolorizing agent include 8.0 g of sodium dithionite (total amount of all reduction agents (a)).

With use of 1.14 g of cocamidopropyl hydroxysultaine, the ratio by weight of (a)/(b) lies at a value of approximately 7.0. With use of 1.29 g of cocamidopropyl hydroxysultaine, the ratio by weight of (a)/(b) lies at a value of approximately 6.2. With use of 1.42 g of cocamidopropyl hydroxysultaine, the ratio by weight of (a)/(b) lies at a value of approximately 5.6. With use of 1.66 g of cocamidopropyl hydroxysultaine, the ratio by weight of (a)/(b) lies at a value of approximately 4.8.

To summarize, it is most generally preferred when the ratio by weight of the total amount of all reduction agents from the group (a) included in the agent to the total amount of all zwitterionic surfactants from the group (b) included in the agent, i.e. the ratio by weight of (a)/(b), lies at a value of from 3.2 to 4.8.

Polyols

It has been found that the use of polyols further supports the decolorization effect. For this reason, it is preferred when the decolorizing agents according to the invention additionally include one or more polyols.

A polyol is understood to mean a compound comprising at least two aliphatic (i.e. not phenolic) OH groups.

Examples of suitable polyols according to the invention are, in particular, ethylene gylcol, 1,2-propylene glycol, 1,3-propanediol, 1,2-butanediol, 1,3-butanediol, 1,2-pentanediol, 1,3-pentanediol, 1,4-pentanediol, 1,5-pentanediol, 1,2-hexanediol, 1,3-hexanediol, 1,4-hexanediol, 1,5-hexanediol and 1,6-hexanediol. Polyethylene glycol and polypropylene glycol, however, are also suitable.

In a further preferred embodiment, a decolorizing agent according to the invention is therefore characterized in that it additionally includes one or more polyols from the group ethylene glycol (1,2-ethanediol), 1,2-propanediol, 1,3-propanediol, 1,2-butanediol, 1,3-butanediol, 1,4-butanediol, 1,2-pentanediol, 1,3-pentanediol, 1,4-pentanediol, 1,5-pentanediol, 1,2-hexanediol, 1,3-hexanediol, 1,4-hexanediol, 1,5-hexanediol and 1,6-hexanediol, polyethylene glycol and/or polypropylene glycol.

The polyols are used in the agents according to the invention preferably in a total amount of from 0.5 to 15.0% by weight, preferably from 2.5 to 13.5% by weight, more preferably from 3.5 to 11.5% by weight, and particularly preferably from 4.5 to 9.5% by weight, in relation to the total weight of the agent.

In a further preferred embodiment, a decolorizing agent according to the invention is therefore characterized in that it additionally includes one or more polyols in a total amount of from 0.5 to 15.0% by weight, preferably from 2.5 to 13.5% by weight, more preferably from 3.5 to 11.5% by weight, and particularly preferably from 4.5 to 9.5% by weight, in relation to the total weight of the agent.

Further Surfactants

Besides the zwitterionic surfactants (b) essential to the invention, the decolorizing agents according to the invention can also include further surfactants, such as non-ionic, ampholytic and/or cationic surfactants. However, it is preferred when the decolorizing agents do not include any anionic surfactants.

In a further preferred embodiment, a decolorizing agent according to the invention is therefore characterized in that the total amount of all anionic surfactants included in the agent lies at a value of at most 0.5% by weight, preferably of at most 0.3% by weight, more preferably of at most 0.1% by weight, and particularly preferably of at most 0.05% by weight, in relation to the total weight of the agent.

The term “anionic surfactants” in the sense of this invention is understood to mean surfactants having exclusively anionic charge.

It is therefore preferred when the total amount of all anionic surfactants included in the agent from the group of
linear fatty acids having 10 to 22 C atoms (soaps)
ethercarboxylic acids of formula R—O—(CH2-CH2O)x-CH2-COOH, in which R is a linear alkyl group having 10 to 22 C atoms and x=0 or is 1 to 16,
acyl sarcosides having 10 to 18 C atoms in the acyl group,
acyl taurides having 10 to 18 C atoms in the acyl group,
acyl isethionates having 10 to 18 C atoms in the acyl group,
sulfosuccinic acid mono- and dialkyl esters having 8 to 18 C atoms in the alkyl group and sulfosuccinic acid monoalkylpolyoxyethyl esters having 8 to 18 C atoms in the alkyl group and 1 to 6 oxyethyl groups,
linear alkanesulfonates having 12 to 18 C atoms,
linear alpha-olefin sulfonates having 12 to 18 C atoms,
alpha-sulfofatty acid methyl esters of fatty acids having 12 to 18 C atoms, and
alkyl sulfates and alkyl polyglycol ether sulfates of formula R—O(CH2-CH2O)x-SO3H, in which R is a preferably linear alkyl group having 10 to 18 C atoms and x=0 or is 1 to 12,
   lies at a value of at most 0.5% by weight, preferably of at most 0.3% by weight, more preferably of at most 0.1% by weight, and particularly preferably of at most 0.05% by weight, in relation to the total weight of the agent.

Non-ionic surfactants are understood to be amphiphilic (bifunctional) compounds comprising at least one hydrophobic group and at least one hydrophilic molecule part. The hydrophobic molecule part is usually a hydrocarbon chain having 10 to 30 carbon atoms. The non-ionic surfactants for example carry a polyol group, a polyalkylene glycol ether group, or a combination of polyol and polyglyol ether group as hydrophilic group. Such compounds are, for example, addition products of from 2 to 30 mol ethylene oxide and/or 0 to 5 mol propylene oxide with linear fatty alcohols having 8 to 22 C atoms, with fatty acids having 12 to 22 C atoms, and with alkyl phenols having 8 to 15 C atoms in the alkyl group, C12-C22 fatty acid mono- and diesters of addition products of from 1 to 30 mol ethylene oxide with glycerol, C8-C22 alkyl mono and oligo glycosides and ethoxylated analogues thereof, and also addition products of from 5 to 60 mol ethylene oxide with castor oil and hardened castor oil.

Preferred non-ionic surfactants are alkyl polyglycosides.

The non-ionic surfactants which can be used in addition can be used in the decolorizing agents according to the invention in a total amount of from 0.1 to 15.0% by weight, preferably from 2.5 to 13.5% by weight, more preferably from 3.5 to 11.5% by weight, and particularly preferably from 4.5 to 9.5% by weight, in relation to the total weight of the agent.

In accordance with the invention, cationic surfactants of the quaternary ammonium compound, esterquat, and amidoamine type are used in particular. Preferred quaternary ammonium compounds are ammonium halides, in particular chlorides and bromides, such as alkyl trimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, and the imidazolium compounds known under the INCI names quaternium-27 and quaternium-83. The long alkyl chains of the above-mentioned surfactants preferably have 10 to 18 carbon atoms. Esterquats are known substances which include both at least one ester function and at least one quaternary ammonium group as structural element. Preferred esterquats and quaternized ester salts of fatty acids with triethanol amine, quaternized ester salts of fatty acids with diethanol alkylamines, and quaternized ester salts of fatty acids with 1,2-dihydroxypropyl dialkylamines. The alkylamidoamines are usually produced by amidation of natural or synthetic fatty acids and fatty acid cuts with dialkyl aminoamines. A particularly suitable compound from this group of substances is the stearamidopropyldimethylamine available commercially under the name Tegoamid® S 18.

Further cationic surfactants that can be used in accordance with the invention are the quaternized protein hydrolyzates.

The cationic surfactants which can be used in addition can be used in the decolorizing agents according to the invention in a total amount of from 0.1 to 15.0% by weight, preferably from 2.5 to 13.5% by weight, more preferably from 3.5 to 11.5% by weight, and particularly preferably from 4.5 to 9.5% by weight, in relation to the total weight of the agent.

Decolorization of Dyed Keratin Fibers

The agent according to the invention is a decolorizing agent which is used for the decolorization of previously dyed keratin fibers, in particular human hair. The dyed keratin fibers are usually fibers that have been previously dyed using conventional oxidation dyes and/or substantive dyes known to a person skilled in the art.

The decolorizing agents are suitable for removing colors produced on the keratin fibers using oxidation dyes on the basis of developer and coupler components. If the following compounds were used as developers, the colors produced as a result can be well removed effectively and practically without subsequent darkening using the decolorizing agent: p-phenylenediamine, p-toluenediamine N,N-bis-(β-hydroxyethyl)-p-phenylenediamine, 4-N,N-bis-(β-hydroxyethyl)-amino-2-methylaniline, 2-(β-hydroxyethyl)-p-phenylenediamine, 2-(α,β-dihydroxyethyl)-p-phenylenediamine, 2-hydroxymethyl-p-phenylenediamine, bis-(2-hydroxy-5-aminophenyl)-methane, p-aminophenol, 4-amino-3-methylphenol, 2,4,5,6-tetraaminopyrimidine, 4-hydroxy-2,5,6-triaminopyrimidine, 2-hydroxy-4,5,6-triaminopyrimidine and/or 4,5-diamino-1-(β-hydroxyethyl)-pyrazole.

If the following compounds were used as couplers, the colors produced as a result can likewise be well removed with very good decolorizing result: m-phenylenediamine derivatives, naphthols, resorcinol and resorcinol derivatives, pyrazolones and m-aminophenol derivatives The following are particularly suitable as coupler substances 1-naphthol, 1,5-, 2,7- and 1,7-dihydroxynaphthaline, 5-amino-2-methylphenol, m-aminophenol, resorcinol, resorcinol monomethyl ether, m-phenylenediamine, 1-phenyl-3-methyl-5-pyrazolone, 2, 4-dichloro-3-aminophenol, 1,3-bi s-(2′,4′-diaminophenoxy) propane, 2-chloro-resorcinol, 4-chloro-resorcinol, 2-chloro-6-methyl-3-aminophenol, 2-amino-3-hydroxypyridine, 2-methylresorcinol, 5-methylresorcinol and 2-methyl-4-chloro-5-aminophenol, 1-naphthol, 1,5-dihydroxynaphthaline, 2,7-dihydroxynaphthaline and 1,7-dihydroxynaphthaline, 3-aminophenol, 5-amino-2-methylphenol, 2-amino-3-hydroxypyridine, resorcinol, 4-chloro-resorcinol, 2-chloro-6-methyl-3-aminophenol, 2-methylresorcinol, 5-methylresorcinol, 2,5-dimethylresorcinol, and 2,6-dihydroxy-3,4-dimethylpyridine.

The substrate to be decolorized can also have been colored using substantive dyes. Substantive dyes include, in particular, nitrophenylenediamine, nitroaminophenols, azo dyes, anthraquinones or indophenols. Preferred substantive dyes are the compounds known under the following international names or trade names: HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, as well as 1,4-diamino-2-nitrobenzene, 2-amino-4-nitrophenol, 1,4-bis-(β-hydroxyethyl)-amino-2-nitrobenzene, 3-nitro-4-(β-hydroxyethyl)-aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamin-2ζ-carboxylic acid, 6-nitro-1,2,3,4-tetrahydroquinoxaline, 2-hydroxy-1,4-naphthoquinone, picraminic acid and salts thereof, 2-amino-6-chloro-4-nitrophenol, 4-ethylamino-3-nitrobenzoic acid and 2-chloro-6-ethylamino-1-hydroxy-4-nitrobenzene.

Furthermore, the substrates to be decolorized can also be dyed using natural dyes occurring in nature, as are included for example in henna red, henna neutral, henna black, chamomile blossom, sandalwood, black tea, cascara bark, sage, logwood, madder root, catechu, sedr, and alkanet root.

The decolorizing agents according to the invention are intended for the removal of these dyed colors and therefore themselves preferably do not include any dyes, i.e. no oxidation dye precursors of the developer type or of the coupler type, and also no substantive dyes.

In a further preferred embodiment a decolorizing agent according to the invention is therefore characterized in that the total amount of all substantive dyes and oxidative dyes included in the agent lies at a value of at most 0.2% by weight, preferably of at most 0.1% by weight, more preferably of at most 0.05% by weight, and particularly preferably of at most 0.01% by weight, in relation to the total weight of the agent.

pH Value and Alkalizing Agent

The previously described decolorizing agents are agents which include the components (a) and (b) essential to the invention in an aqueous cosmetic carrier. The pH value of the decolorizing agent can be set in principle to values between 2 and 12. For reasons of stability and for the purpose of storage, however, it is advantageous when the agent has an alkaline pH value. The agent is therefore preferably set to a pH value of from 7 to 12, more preferably 7.5 to 11.5, even more preferably from 8.0 to 11.0, and very particularly preferably from 8.5 to 10.5.

The pH values were measured using a glass electrode of the N 61 type from the company Schott at a temperature of 22° C.

In a further preferred embodiment a decolorizing agent according to the invention is therefore characterized in that it has a pH value of from 7.0 to 12.0, preferably from 7.5 to 11.5, even more preferably from 8.0 to 11.0, and very particularly preferably from 8.5 to 10.5 (measured using a glass electrode of the N 61 type from the company Schott at a temperature of 22° C.).

The alkalizing agents usable in accordance with the invention to set the preferred pH value can be formed from the group of ammonia, alkanolamines, basic amino acids, and inorganic alkalizing agents, such as alkaline (earth) metal hydroxides, alkaline (earth) metal metasilicates, alkaline (earth) metal phosphates, and alkaline (earth) metal hydrogen phosphates. Suitable inorganic alkalizing agents are sodium hydroxide, potassium hydroxide, sodium silicate, and sodium metasilicate. Organic alkalizing agents that can be used in accordance with the invention can be selected from monoethanolamine, 2-amino-methylpropanol, and triethanolamine. The basic amino acids that can be used as alkalizing agent according to the invention can be selected from the group formed from arginine, lysine, ornithine and histidine.

The decolorizing agents according to the invention are particularly preferably brought to an alkaline pH value by use of one or more alkalizing agents from the group of sodium hydroxide, potassium hydroxide, ammonia, monoethanolamine and/or arginine. Sodium hydroxide and/or potassium hydroxide are very particularly preferably selected from this group.

In a further preferred embodiment a decolorizing agent according to the invention is therefore characterized in that it additionally includes one or more alkalizing agents selected from the group of sodium hydroxide, potassium hydroxide, ammonia, monoethanolamine and/or arginine, particularly preferably selected from the group of sodium hydroxide and/or potassium hydroxide.

Gels

Depending on the dyes used previously for coloring and depending on the condition, the degree of damage, and the thickness of the hair, the decolorizing process can last for a different amount of time for different individuals. Although the decolorizing process should generally be complete within the usual application times of up to 60 minutes, it is much more comfortable for the user of the decolorizing agent to directly monitor the decolorizing process and to be able to rinse off the decolorizing agent immediately after completion of the decolorization, possibly after just 20 or 30 minutes.

The decolorizing process can be directly monitored by the user or hairdresser when the decolorizing agent is provided in the form of a transparent gel.

In a further preferred embodiment, a decolorizing agent according to the invention is therefore characterized in that it is a clear, flowable gel formulation having a transmittance T of at least 70%, preferably of at least 75%, more preferably of at least 80%, and particularly preferably of at least 85%, wherein the transmittance T is calculated by the following formula

T=Φex/Φin

with
Φex equal to the radiation intensity of the light beam that has passed through and exits from the agent, and
Φin is equal to the radiation intensity of the light beam passed into the agent.

Gels are understood to mean systems which consist of a solid, colloidally distributed substance, which is a thickening agent or a gelling agent, and a liquid (water or water-solvent mixtures). Here, the thickening agent or gelling agent forms a physical network in the liquid.

The gels in the sense of the present invention are flowable, which means that they preferably have a viscosity of from 1,000 mPas to 15,000 mPas, particularly preferably of 3,000 mPas to 8,000 mPas (with measurements taken using a rotary viscosimeter from Brookfield, spindle size 4, at 25° C. and 20 rpm).

The transmission describes the permeability of the gel for the measurement light (preferably daylight) and is expressed as transmittance T. The transmittance constitutes a ratio value T=Φex/Φin, wherein the radiation intensity of the light beam that has passed through and exits from the agent (Φex) is set in relation to the radiation intensity of the light beam passed into the agent (Φin). The measurement is taken with daylight (daylight lamp) with a layer thickness of 1 cm (i.e. the gel to be measured is filled into a tank so that the gel is present in a layer thickness of 1 cm and is then measured using a commercially available photometer).

With a transmittance of at least 70%, preferably of at least 75%, more preferably of at least 80%, and particularly preferably at least 85%, the gel is so transparent that the consumer can observe the decolorizing process of the hair directly through the transparent gel applied to the hair. In this way, the consumer can visually perceive the end of the decolorizing process directly, without having to rinse off the decolorizing agent from a test strand of the treated hair.

By way of example, agar-agar, carrageenan, alginates, xanthan gum, karaya gum, gum ghatti, tragacanth, scleroglucan or gum arabic, alginates, pectins, polyoses, guar gum, carob bean gum, linseed gums, dextrans, pectins, starch fractions and derivatives such as amylose, amylopectin and dextrins, gelatin and casein, and cellulose derivatives such as methylcellulose, carboxyalkylcelluloses such as carboxymethylcellulose, and hydroxyalkylcelluloses such as hydroxyethyl cellulose can be used as gelling agents or thickening agents to produce the transparent gel.

With use of xanthan, the decolorizing agents can be provided in the form of flowable gels, of which the viscosity is optimized in respect of the application process and which enable a direct visual assessment of the decolorizing process particularly well. For this reason, the use of xanthan gel thickening agents is particularly preferred.

In a further preferred embodiment a decolorizing agent according to the invention is therefore characterized in that it additionally includes xanthan as thickening agent.

Kit-of-Parts

As previously described, the decolorizing agent can be set preferably to an alkaline pH value for the purpose of storage, since it has improved stability at alkaline pH values. The decolorizing effect of the combination of reduction agent (a) and zwitterionic surfactant (b), however, is a function of the pH value, which experiences its optimum in acid medium. For this reason, it is advantageous to bring the agent according to the invention to an acid pH value just before application, said pH value preferably lying in a range of 4 to 6. The pH value of the previously alkaline agent should therefore be lowered just before application

The acidification of the previously alkaline decolorizing agent just before application can be implemented by the mixing of two different agents, wherein the previously described alkaline first agent is mixed with a further agent which includes one or more acids.

By way of example, one or more acids from the group of citric acid, tartaric acid, malic acid, lactic acid, acetic acid, sulfuric acid, hydrochloric acid, phosphoric acid, methanesulfonic acid, benzoic acid, malonic acid, oxalic acid and/or 1-hydroxyethane-1,1-diphosphonic acid can be used as acids.

A second subject matter of the present invention is a multi-component packaging unit (kit-of-parts) for the reductive decolorization of dyed keratinic fibers, which unit comprises, packaged separately from one another

(I) in a container (I) an agent (A) and
(II) in a container (II) an agent (B), wherein
the agent (A) in container (I) is an agent as described herein, and
the agent (B) in container (II) is a cosmetic agent containing at least one organic and/or inorganic acid from the group of citric acid, tartaric acid, malic acid, lactic acid, acetic acid, sulfuric acid, hydrochloric acid, phosphoric acid, methanesulfonic acid, benzoic acid, malonic acid, oxalic acid and/or 1-hydroxyethane-1,1-diphosphonic acid.

In other words, the ready-to-use decolorizing agent is produced by mixing agent (A) from container (I) and agent (B) from container (II).

The agent (A) forming the first subject matter of the invention is the preferably alkaline and storage-stable agent containing the reduction agent(s) (a) and also the zwitterionic surfactants (b). By mixing agent (A) with the one or more acid-containing agent(s) (B), the pH value of the ready-to-use agent is lowered and is brought to the value optimal for application.

The acids citric acid, tartaric acid, malic acid, lactic acid, methanesulfonic acid, malonic acid, oxalic acid and/or 1-hydroxyethane-1,1-diphosphonic acid have proven to be particularly suitable.

The agents (B) in container (II), which include at least one acid, can be provided in solid or liquid form. The agent (B) in container (II) is preferably a liquid agent with a liquid carrier.

The agent (B) in container (II) preferably has a pH value of from 1 to 6, preferably from 1.3 to 4.5, more preferably from 1.6 to 4.0, and particularly preferably from 2.0 to 3.6, wherein these pH values can be measured for example using a glass electrode of the N61 type from the company Schott at a temperature of 22° C.

A multi-component packaging unit (kit-of-parts) in which
the agent (B) in container (II) is an agent which includes in an aqueous cosmetic carrier at least one organic acid from the group of citric acid, tartaric acid, malic acid, lactic acid, methanesulfonic acid, malonic acid, oxalic acid and/or 1-hydroxyethane-1,1-diphosphonic acid and has a pH value of from 1 to 6, preferably from 1.3 to 4.5, more preferably from 1.6 to 4.0, and particularly preferably from 2.0 to 3.6 (measured using a glass electrode of the N61 type from the company Schott at a temperature of 22° C.) is therefore particularly preferred.

The multi-component packaging unit (kit-of-parts) can also optionally comprise a further, third separately packaged container (III), wherein the container (III) includes an agent (C) which can be a shampoo or a conditioner.

A multi-component packaging unit (kit-of-parts) for the reductive decolorization of dyed keratinic fibers which comprises, packaged separately from one another,

(I) in a container (I) an agent (A) and
(II) in a container (II) an agent (B),
(III) in a container (III) an agent (C), wherein
the agent (A) in container (I) is an agent forming the first subject matter of the invention
the agent (B) in container (II) is a cosmetic agent containing at least one organic and/or inorganic acid from the group of citric acid, tartaric acid, malic acid, lactic acid, acetic acid, sulfuric acid, hydrochloric acid, phosphoric acid, methanesulfonic acid, benzoic acid, malonic acid, oxalic acid and/or 1-hydroxyethane-1,1-diphosphonic acid,
the agent (C) in container (III) is a cosmetic agent containing one or more zwitterionic surfactants
   is therefore also preferred.

Likewise, a multi-component packaging unit (kit-of-parts) for the reductive decolorization of dyed keratinic fibers which comprises, packaged separately from one another,

(I) in a container (I) an agent (A) and
(II) in a container (II) an agent (B),
(III) in a container (III) an agent (C), wherein
the agent (A) in container (I) is an agent forming the first subject matter of the invention
the agent (B) in container (II) is a cosmetic agent containing at least one organic and/or inorganic acid from the group of citric acid, tartaric acid, malic acid, lactic acid, acetic acid, sulfuric acid, hydrochloric acid, phosphoric acid, methanesulfonic acid, benzoic acid, malonic acid, oxalic acid and/or 1-hydroxyethane-1,1-diphosphonic acid,
the agent (C) in container (III) is a cosmetic agent containing one or more zwitterionic surfactants, each of which has at least one quaternary ammonium group and a grouping of —SO₃— as structural units, is also preferred.

Mixing Ratio of Agents (A) and (B)

As already described beforehand, the ready-to-use decolorizing agent is preferably produced by mixing agents (A) and (B). In principle, the agents (A) and (B) can be mixed here in different mixing ratios, such as (A)/(B) from 20:1 to 1:20.

In order to ensure comfortable mixing, it may be advantageous to use the two agents (A) and (B) in approximately equal amounts.

In particular if the acids included in the agent (B) are used in concentrated form, it may also be advantageous, however, to use the agent (A) in a surplus.

In a further preferred embodiment a multi-component packaging unit according to the invention is therefore characterized in that the amounts of the agent (A) in container (I) and of the agent (B) in container (II) are selected such that, when producing the application mixture—i.e. when mixing the agents (A and (B)—the mixing ratio (A)/(B) lies at a value of at least 1, preferably at least 1.3, more preferably at least 1.5, and particularly preferably at least 2.0.

In order to produce the mixture, the agent (A) from container (I) can, for example, be transferred completely into container (II), which already includes the agent (B). In this case the size of the container (II) is selected such that the container (II) can receive the total amount of agent (A) and (B) and also allows the two agents (A) and (B) to be mixed, for example by shaking or stirring.

Similarly, the mixture can also be produced by completely transferring the agent (B) from container (II) into container (I), which already includes the agent (A). In this case the size of the container (I) should be selected such that the container (I) can receive the total amount of agent (A) and (B) and also allows the two agents (A) and (B) to be mixed, for example by shaking or stirring.

A further possibility for producing the application mixture is the complete transfer of both agents (A) and (B) from containers (I) and (II) into a third container, which then allows the two agents to be mixed, for example by shaking or stirring.

### Example

A multi-component packaging unit according to the invention includes
100 g of agent (A) in container (I)
50 g of agent (B) in container (II).

In order to produce the application mixture, agent (B) from container (II) is transferred completely into container (I). The agents (A) and (B) are then shaken or stirred with one another. The mixing ratio of agents (A)/(B) then lies at a value of (100 g/50 g)=2.0.

Further Ingredients

The agents (A) and (B) and, where applicable, (C) can also include additional active substances, auxiliaries and additives in order to improve the decolorizing power and adjust further desired properties of the agent. By way of example, one or more of the agents can additionally include non-ionic polymers, such as vinylpyrrolidone/vinylacrylate copolymers, polyvinylpyrrolidone, vinylpyrrolidone/vinylacetate copolymers, polyethylene glycols and polysiloxanes; additional silicones such as volatile or non-volatile, straight-chain, branched or cyclic, cross-linked or un-cross-linked poly alkyl siloxanes (such as dimethicones or cyclomethicones), poly aryl siloxanes and/or polyalkyl aryl siloxanes, in particular polysiloxanes having organofunctional groups, such as substituted or unsubstituted amines (amodimethicones), carboxyl, alkoxy and/or hydroxyl groups (dimethicone copolyols), linear polysiloxane (A)-polyoxyalkylene (B) block copolymers, grafted silicone polymers; cationic polymers such as quaternized cellulose ethers, polysiloxanes with quaternary groups, dimethyl diallyl ammonium chloride polymers, acrylamide-dimethyl diallyl ammonium chloride copolymers, dimethylaminoethyl methacrylate-vinyl pyrrolidone copolymers quaternized with diethyl sulfate, vinyl pyrrolidone-imidazolinium-methochloride copolymers, and quaternized polyvinyl alcohol; zwitterionic and amphoteric polymers; anionic polymers such as polyacrylic acids or cross-linked polyacrylic acids; structuring substances such as glucose, maleic acid and lactic acid, hair-conditioning compounds such as phospholipids, for example lecithin and cephalin, dimethyl isosorbide and cyclodextrins; fiber structure-improving active substances, in particular mono, di, and oligosaccharides, for example glucose, galactose, fructose, fruit sugars and lactose; dyes for colouring the agent; anti-dandruff active substances such as piroctone olamine, zinc omadine and climbazole; amino acids and oligopeptides; animal-based and/or plant-based protein hydrolyzates and protein hydrolyzates in the form of their fatty acid condensation products or optionally anionically or cationically modified derivatives; vegetable oils; light stabilizers and UV blockers; active substances such as panthenol, pantothenic acid, pantolactone. allantoin, pyrrolidone carboxylic acids and salts thereof, and bisabolol; polyphenols, in particular hydroxycinnamic acids, 6,7-dihydroxycoumarin, hydroxybenzoic acids, catechins, tannins, leukoanthocyanidins, anthocyanidins, flavanones, flavones and flavonols; ceramides or pseudoceramides; vitamins, pro-vitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, ureas and primary, secondary and tertiary phosphates; turbidity agents such as latex, styrene/PVP and styrene/acrylamide copolymers; pearlescent agents such as ethylene glycol mono- and distearate and PEG-3 distearate; pigments as well as propellants such as propane-butane mixtures, N20, dimethyl ether, CO₂ and air. Express reference is made in this regard to the known monographs, for example Kh. Schrader, Grundlagen and Rezepturen der Kosmetika (Basic Principles and Formulations of Cosmetics), 2^{nd} edition, Hüthig Buch publishers, Heidelberg, 1989, which reflect the corresponding knowledge of a person skilled in the art.

Method

The previously described agents and multi-component packaging units (kits-of-parts) according to the invention can be used in methods for dyeing and reductive decolorization.

A third subject matter of the present invention is therefore a method for the dyeing and reductive decolorization of keratinic fibers, comprising the following steps in the specified order

(I) applying a cosmetic coloring agent, which includes at least one substantive dye and/or at least one oxidation dye precursor, to the keratinic fibers,
(II) leaving the coloring agent to act for a period of time lasting from 5 to 60 minutes,
(III) rinsing out the coloring agent,
(IV) producing a ready-to-use decolorizing agent by mixing an agent forming the first subject matter of the invention with an agent which includes at least one organic and/or inorganic acid from the group of citric acid, tartaric acid, malic acid, lactic acid, acetic acid, sulfuric acid, hydrochloric acid, phosphoric acid, methanesulfonic acid, benzoic acid and/or 1-hydroxyethane-1,1-diphosphonic acid,
(V) applying the ready-to-use decolorizing agent (obtained in step (IV) to the keratinic fibers,
(VI) leaving the decolorizing agent to act for a period of time lasting from 5 to 60 minutes, preferably from 10 to 55 minutes, more preferably from 15 to 55 minutes, and particularly preferably from 20 to 50 minutes,
(VII) rinsing out the decolorizing agent,
(VIII) optionally applying a post-treatment agent to the keratinic fibers, wherein the post-treatment agent includes at least one surfactant.

The steps (I), (II) and (III) of the method constitute the dyeing process of the keratin fibers and are consequently performed in direct chronological succession after one another. For the sequence of steps (III) and (IV), there is in principle no time limitation. Step (IV) can thus be carried out hours, days or also for example up to two weeks after completion of step (III).

The method, however, is intended to remove the undesired color result of the dyeing process of steps (I) to (III), and it therefore goes without saying that the decolorization can only be performed when the dyed fibers still present the undesired color result. If the keratin fibers were dyed for example using substantive dyes, and if this coloration has already completely washed out after 2 weeks, a decolorizing process taking place subsequently is neither necessary nor proposed in accordance with the invention.

In step (IV) of the method according to the invention a ready-to-use decolorizing agent is produced by mixing an agent forming the first subject matter of the invention with a further agent containing at least one of the aforementioned acids. Steps (IV), (V), (VI) and (VII) of the method constitute the decolorizing process of the keratin fibers and are consequently carried out again in direct chronological succession one after the other.

Step (VIII) of the method, i.e. the application of a post-treatment agent, is optional. There is again no time limitation for the sequence of steps (VII) and of optional step (VIII).

It is, however, advantageous when the post-treatment of step (VIII) is performed at most two days after completion of step (VII). The post-treatment step (VIII) can also be repeated more frequently than once, for example when the post-treatment agent is a shampoo.

During the course of the works leading to this invention, it was found that a post-treatment in step (VIII) with a post-treatment agent containing one or more zwitterionic surfactants, each of which has at least one quaternary ammonium group and a grouping of —SO₃—, is of very particular advantage.

When the post-treatment agent applied in step (VIII) includes a zwitterionic surfactant (b), similarly to the decolorizing agent, the inhibition of the re-oxidation, i.e. the prevention of the darkening, is maintained for such a length of time that it even lasts for a number of hair washes. This is the case in particular when the post-treatment agent applied in step (VIII) is a shampoo which is used routinely after the decolorization.

A method for the dyeing and reductive decolorization of keratinic fibers, comprising the following steps in the specified order

(I) applying a cosmetic coloring agent, which includes at least one substantive dye and/or at least one oxidation dye precursor, to the keratinic fibers,
(II) leaving the coloring agent to act for a period of time lasting from 5 to 60 minutes,
(III) rinsing out the coloring agent,
(IV) producing a ready-to-use decolorizing agent by mixing an agent forming the first subject matter of the invention with an agent which includes at least one organic and/or inorganic acid from the group of citric acid, tartaric acid, malic acid, lactic acid, acetic acid, sulfuric acid, hydrochloric acid, phosphoric acid, methanesulfonic acid, benzoic acid and/or 1-hydroxyethane-1,1-diphosphonic acid,
(V) applying the ready-to-use decolorizing agent from step (IV) to the keratinic fibers,
(VI) leaving the decolorizing agent to act for a period of time lasting from 5 to 60 minutes, preferably from 10 to 55 minutes, more preferably from 15 to 55 minutes, and particularly preferably from 20 to 50 minutes,
(VII) rinsing out the decolorizing agent,
(VIII) applying a post-treatment agent to the keratinic fibers, wherein the post-treatment agent includes one or more zwitterionic surfactants, each of which has at least one quaternary ammonium group and a grouping of —SO₃— as structural units is therefore also very particularly preferred.

Use

Within the scope of the works leading to this invention, it has been found that zwitterionic surfactants which each have at least one quaternary ammonium group and a grouping of —SO₃— as structural units can effectively prevent the darkening of reductively dyed keratin fibers, moreover in a long-lasting manner.

Preferred and particularly preferred zwitterionic surfactants can be cited here as the surfactants (b) which have also been referred to as preferred and particularly preferred in the embodiments relating to the first subject matter of the invention.

A further subject matter of the present invention is therefore the use of a zwitterionic surfactant which has at least one quaternary ammonium group and one grouping of —SO₃— as structural units, to improve the decolorizing effect of one or more reduction agents from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, sodium disulfite, potassium disulfate, ammonium disulfite, hydroxymethanesulfinic acid, aminomethanesulfinic acid, cysteine, thiolactic acid, sulfanylacetic acid (thioglycolic acid) and/or ascorbic acid in the reductive decolorization of dyed keratinic fibers.

That said in relation to the agents according to the invention applies mutatis mutandis with regard to further preferred embodiments of the multi-component packaging unit (kit-of-parts) according to the invention, the method according to the invention, and the use according to the invention.

### EXAMPLES

1.1. Coloring

The following formulations were produced (all values in % by weight):

Coloring cream (F1)

| | % by |
|---|---|
| Raw material | weight |
| Cetearyl alcohol | 8.5 |
| C12-C18 fatty alcohols | 3.0 |
| Ceteareth-20 | 0.5 |
| Ceteareth-12 | 0.5 |
| Plantacare 1200 UP | 2.0 |
| (Laurylglucoside, 50-53% | |
| aqueous solution) | |
| Sodium laureth-6 carboxylate | 10.0 |
| (21% aqueous solution) | |
| Sodiummyreth sulfate | 2.8 |
| (68-73% aqueous solution) | |
| Sodium acrylate, | 3.8 |
| trimethylammoniopropylacrylamide | |
| chloride copolymer | |
| (19-21% aqueous solution) | |
| Potassium hydroxide | 0.83 |
| p-toluenediamine, sulfate | 2.25 |
| m-aminophenol | 0.075 |
| 2-amino-3-hydroxypyridine | 0.12 |
| Resorcinol | 0.62 |
| 4-chlororesorcinol | 0.26 |
| 3-amino-2-methylamino-6-methoxypyridine | 0.04 |
| 1,3-bis(2,4-diaminophenoxy)propane, | 0.05 |
| tetrahydrochloride | |
| Ammonium sulfate | 0.1 |
| Sodium sulfite | 0.4 |
| Ascorbic acid | 0.1 |
| 1-hydroxyethan-1,1-diphosphonic acid | 0.2 |
| (60% aqueous solution) | |
| Ammonia (25% aqueous solution) | 7.2 |
| Water | to 100 |

Oxidizing agent (Ox)

| | % by |
|---|---|
| Raw material | weight |
| Sodium benzoate | 0.04 |
| Dipicolinic acid | 0.1 |
| Disodiumpyrophosphate | 0.1 |
| Potassium hydroxide | 0.09 |
| 1,2-propylene glycol | 1.0 |
| 1-hydroxyethan-1,1-diphosphonic acid | 0.25 |
| (60% aqueous solution) | |
| Paraffinum liquidum | 0.30 |
| Steartrimonium chloride | 0.39 |
| Cetearyl alcohol | 3.4 |
| Ceteareth-20 | 1.0 |
| Hydrogen peroxide | 12.0 |
| (50% aqueous solution) | |

The coloring cream (F1) and the oxidizing agent (Ox) were mixed in a proportion of 1:1 and applied to strands of hair (Kerling natural European hair, white). The ratio by weight of application mixture:hair was 4:1, and the reaction time was 30 minutes at a temperature of 32 degrees Celsius. The strands were then rinsed with water, dried, and left to rest for at least 24 hours at room temperature. The strands were dyed to a dark brown shade.

1.2. Decolorization

The following decolorizing agents were produced (all values in % by weight of active substance):

| | V1 | E1 |
|---|---|---|
| | (comparison) | (invention) |
| Sodium dithionite | 8.8 | 8.8 |
| Cocamidopropyl | — | 2.5 |
| Sultaine | | |
| Sodium hydroxide | 0.5 | 0.5 |
| Xanthan | 1.5 | 1.5 |
| Propylene glycol | 6.0 | 6.0 |

The pH value of the two formulations V1 and E1 was between 8 and 10.

For application, each of the decolorizing agents V1 and E1 was set to a pH value of from 4 to 5 by addition of an aqueous citric acid solution.

This ready-to-use decolorizing agent was applied to the colored hair under point 1.1 and left to act for 45 minutes at a temperature of 30° C. The strands were then rinsed out with water for 2 minutes and dried.

The coloration of the strands was assessed visually depending on time. The assessment of color intensity was made on the basis of then following scale:

0—strand no longer has any perceivable coloration (white-blonde, similarly to the original coloration of the used Kerling natural European hair, white)
1—strand colored with weak color intensity
2—strand colored with medium color intensity
3—strand colored with heavy color intensity
4—coloration of the strands similar to that directly after the dyeing, no decolorization effect

| | V1 | E1 |
|---|---|---|
| | (comparison) | (invention) |
| Coloration of the strands as | 0 | 0 |
| the decolorization agent is | | |
| being washed out | | |
| Coloration of the strands | 2 | 1 |
| directly after washing out | | |
| Coloration of the strands | 3 | 1 |
| after 10 minutes | | |
| Coloration of the strands | 3 | 1 |
| after 60 minutes | | |
| Coloration of the strands | 3 | 2 |
| after 1 day | | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An agent for the reductive decolorization of dyed keratinic fibers, in particular human hair, comprising, in an aqueous cosmetic carrier
(a) one or more reduction agents selected from the group of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, thioglycolic acid and ascorbic acid, and
(b) one or more zwitterionic surfactants, each of which has at least one quaternary ammonium group and at least one grouping of —SO₃— as structural units.

2. The agent according to claim 1, wherein the agent includes 0.5 to 20.5% by weight of (a) one or more reduction agents selected from the group consisting of sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate and ammonium thiosulfate.

3. The agent according to claim 1, wherein the agent includes 0.1 to 15.0% by weight, in relation to the total weight of the agent, as zwitterionic surfactant (b), one or more surfactants of formula (I) 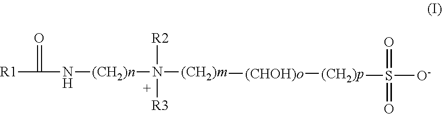
in which
R1 stands for a linear or branched C₉-C₂₉ alkyl group, a linear or branched C₉-C₂₉ alkenyl group, or a linear or branched hydroxy C₉-C₂₉ alkyl group,
R2 and R3 independently of one another, stand for a C₁-C₆ alkyl group, a C₂-C₆ alkenyl group, or a hydroxy C₂-C₆ alkyl group,
n stands for an integer from 1 to 6,
m stands for an integer from 0 to 6,
o stands for an integer from 0 to 6,
p stands for an integer from 0 to 6,
with the provision that the sum of m, o and p is at least 1.

4. The agent according to claim 1, wherein the ratio by weight of the total amount of all reduction agents from the group (a) included in the agent to the total amount of all zwitterionic surfactants from the group (b) included in the agent, is 1.0 or less.

5. The agent according to claim 1, wherein the ratio by weight of the total amount of all reduction agents from the group (a) included in the agent to the total amount of all zwitterionic surfactants from the group (b) included in the agent is 7 or less.

6. The agent according to claim 1, wherein the ratio by weight of the total amount of all reduction agents from the group (a) included in the agent to the total amount of all zwitterionic surfactants from the group (b) included in the agent is 4.8 or less.

7. The agent according to claim 1, wherein the agent further comprises 0.5% to 15.0% by weight, based on the total weight of the agent, of one or more polyols.

8. The agent according to claim 1, wherein the agent further comprises 4.5 to 9.5% by weight, based on the total weight of the agent, of one or more polyols.

9. The agent according to claim 1, wherein the agent includes up to 0.5% by weight, based on the total weight of the agent, of anionic surfactants.

10. The agent according to claim 1, wherein the agent includes up to 0.1% by weight, based on the total weight of the agent, of anionic surfactants.

11. The agent according to claim 1, wherein the agent includes up to 0.2% by weight, based on the total weight of the agent, of all substantive dyes and oxidative dyes.

12. The agent according to claim 1, wherein the agent includes up to 0.1% by weight, based on the total weight of the agent, of all substantive dyes and oxidative dyes.

13. The agent according to claim 1, wherein the agent includes up to 0.01% by weight, based on the total weight of the agent, of all substantive dyes and oxidative dyes.

14. The agent according to any claim 1, wherein the agent has a pH value of 7.0 to 12.0.

15. The agent according to any claim 1, wherein the agent has a pH value of 8.5 to 10.5.

16. The agent according to claim 1, wherein the agent further comprises one or more alkalizing agents selected from the group of sodium hydroxide, potassium hydroxide, ammonia, monoethanolamine, and/or arginine, preferably selected from the group of sodium hydroxide and potassium hydroxide.

17. The agent according to claim 1, wherein the agent is a clear, flowable gel formulation having a transmittance T of at least 70%, wherein the transmittance T is calculated via the following formula
T=Φex/Φin
with
Φex equal to the radiation intensity of the light beam that has passed through and exits from the agent, and
Φin is equal to the radiation intensity of the light beam that passes into the agent.

18. A multi-component packaging unit (kit-of-parts) for the reductive decolorization of dyed keratinic fibers, which unit comprises, packaged separately from one another
(I) in a container (I) an agent (A) and
(II) in a container (II) an agent (B), wherein
the agent (A) in container (I) is an agent according to claim 1 and
the agent (B) in container (II) is a cosmetic agent including at least one organic and/or inorganic acid selected from the group consisting of citric acid, tartaric acid, malic acid, lactic acid, acetic acid, sulfuric acid, hydrochloric acid, phosphoric acid, methanesulfonic acid, benzoic acid, malonic acid, oxalic acid and 1-hydroxyethane-1,1-diphosphonic acid.

19. The multi-component packaging unit (kit-of-parts) according to claim 13, wherein—the agent (B) in container (II) is an agent which includes in an aqueous cosmetic carrier, at least one organic acid selected from the group consisting of citric acid, tartaric acid, malic acid, lactic acid, methanesulfonic acid, malonic acid, oxalic acid and 1-hydroxyethane-1,1-diphosphonic acid, and has a pH value of from 1 to 6.

20. A method for the dyeing and reductive decolorization of keratinic fibers, comprising the following steps in the specified order
(I) applying a cosmetic coloring agent, which includes at least one substantive dye and/or at least one oxidation dye precursor, to the keratinic fibers,
(II) leaving the coloring agent to act for a period of time lasting from 5 to 60 minutes,
(III) rinsing out the coloring agent,
(IV) producing a ready-to-use decolorizing agent by mixing an agent according to claim 1 with an agent which includes at least one organic and/or inorganic acid selected from the group consisting of citric acid, tartaric acid, malic acid, lactic acid, acetic acid, sulfuric acid, hydrochloric acid, phosphoric acid, methanesulfonic acid, benzoic acid and/or 1-hydroxyethane-1,1-diphosphonic acid,
(V) applying the ready-to-use decolorizing agent to the keratinic fibers,
(VI) leaving the decolorizing agent to act for a period of time lasting from 5 to 60 minutes,
(VII) rinsing out the decolorizing agent, and
(VIII) optionally applying a post-treatment agent to the keratinic fibers, wherein the post-treatment agent includes at least one amphoteric and/or zwitterionic surfactant.
